(19) United States
(12) Patent Application Publication
(10) Pub. No.: US 2010/0061934 A1
(43) Pub. Date: Mar. 11, 2010

(54) Methods for diagnosis or treatment of amyloidosis by targeting hyper-sulfated proteoglycans present in amyloid

(76) Inventors: Jonathan Wall, Knoxville, TN (US); Stephen Kennel, Kingston, TN (US); Toin van Kuppevelt, Nijmegen (NL); Nicole Smits, Lebanon, NH (US)

Correspondence Address: HOWARD EISENBERG, ESQ., 1220 LIMBERLOST LANE, GLADWYNE, PA, 19035, US

(21) Appl. No.: 12/584,522

(22) Filed: Sep. 8, 2009

Related U.S. Application Data
(60) Provisional application No. 61/191,320, filed on Sep. 8, 2008.

(51) Int. Cl. A61K 39/395; A61K 49/00

(52) U.S. Cl. 424/9.1; 424/137.1

(57) ABSTRACT

Amyloid in an individual may be targeted by systemically administering to an individual a ligand that binds to a hyper-sulfated proteoglycan and permitting the ligand to bind to hyper-sulfated proteoglycans associated with amyloid within the body of the individual.

This application claims priority from U.S. Provisional patent application Ser. No. 61/191,320, filed Sep. 8, 2008, which application is incorporated herein in its entirey by reference.

### FIELD OF THE INVENTION

The present application pertains to the field of targeting amyloid for diagnostic and/or therapeutic purposes.

### BACKGROUND

Amyloidosis refers to the pathological deposition of proteins in the form of congophilic, green birefringent fibrils, when Congo red-stained, either dispersed or in the form of localized amyloidomas. Such deposits are symptomatic of several diseases, for example Alzheimer's Disease, inflammation-associated amyloid, type II diabetes, bovine spongiform encephalopathy (BSE), Creutzfeld-Jakob disease (CJD), scrapie, and primary amyloidosis.

Amyloidoses are generally categorized into three groups: major systemic amyloidoses, major localized amyloidoses, and miscellaneous amyloidoses. Major systemic amyloidoses include: chronic inflammatory conditions (e.g., tuberculosis, osteomyelitis, etc.); non-infectious conditions such as juvenile rheumatoid arthritis, ankylosing spondylitis and Crohn's disease, etc.; familial Mediterranean Fever, plasma cell dyscrasia (primary amyloidosis) and various familial polyneuropathies and cardiomyopathies. Major localized amyloidoses include: chronic dialysis usually for greater than 8 years, Alzheimer's disease, Down syndrome, Hereditary cerebral hemorrhage (Dutch), and non-traumatic cerebral hemorrhage of the elderly.

Miscellaneous amyloidoses include: familial polyneuropathy (Iowa), familial amyloidosis (Finnish), hereditary cerebral hemorrhage (Icelandic), CJD, Medullary carcinoma of the thyroid, atrial amyloid, and diabetes mellitus (insulinomas). Other amyloidoses include those referenced in Louis W. Heck, “The Amyloid Diseases” in Cecil's Textbook of Medicine 1504-6 (W. B. Saunders & Co., Philadelphia, Pa.; 1996).

Transmissible spongiform encephalopathies which cause CJD and Gerstmann-Strassler-Scheinker (GSS) disease are described by B. Chesebro et al., “Transmissible Spongiform Encephalopathies: A Brief Introduction” in FIELD'S VIROLOGY 2845-49 (3rd Edition; Raven Publishers, Philadelphia, Pa.; 1996) and in D.C. Gajdusek,“Infectious amyloids: Subacute Spongiform Encephalopathies as Transmissible Cerebral Amyloidoses,”2851-2900 in FIELDS VIROLOGY (1996). Many of these diseases are likely mediated by prions, an infectious protein. See S. B. Prusiner, “Prions” in FIELDS VIROLOGY 2901-50 (1996) and the references contained therein.

One problem that is encountered in the field of therapy of amyloid-related disorders is the inability to image amyloid in a patient suffering therefrom. To date, no satisfactory method has been developed by which it is possible to obtain a quantitative, complete, image of amyloid deposition in a body. Consequently, it is difficult, if even possible, to monitor response to therapy for amyloidosis in a live subject. Instead, post-mortem analysis of the extent of amyloid is currently the means by which response to therapy for amyloidosis is evaluated, or by using surrogate markers of organ function as presumed and indirect measure of amyloid burden. A significant need exists for a method by which an ante-mortem determination of the degree and extent of amyloidosis may be obtained. Such a method would permit the assessment of extent and prognosis of amyloidosis in an individual and would permit the rational evaluation of therapeutic response against amyloidosis.

Another problem that is encountered in the field of therapy of amyloid-related disorders is the lack of effective treatment modalities. Solomon et al, WO 99/60024 discloses a method for amyloid removal using antibodies that specifically bind to the fibrillar constituents of amyloid. In many situations, however, an antibody that binds to the protein fibrillar constituents of amyloid associated with a particular condition may not bind to the protein constituents of amyloid associated with other conditions. Consequently, a significant need exists for a treatment modality for amyloidosis that can be utilized against amyloidosis associated with any disorder.

Proteoglycans, also referred to as glycosaminoglycans, are nonfibrillar components that are present in amyloid associated with all amyloid-related disorders. They are composed of long unbranched polysaccharides that contain distinct repeating disaccharide units. Many types of proteoglycans exist in amyloid. Examples of proteoglycans that are present in amyloid include heparan sulfate, dermatan sulfate, and chondroitin sulfate.

Sulfated proteoglycans, such as heparan sulfate, dermatan sulfate, and chondroitin sulfate, are present in normal tissues throughout the body that are free of amyloid. The sulfated proteoglycans found normally in the body contain disaccharide units that are mono-sulfated or di-sulfated. Sulfated proteoglycans that contain disaccharide units that contain more than two sulfate groups, referred to herein as “hyper-sulfated,” are rare in normal tissues but have been determined to be present in amyloid. Regarding dermatan sulfate and chondroitin sulfate, these sulfated proteoglycans are considered to be “hyper-sulfated” if they contain two or more sulfate groups, such as at any two of positions 2, 4, and 6.

### DESCRIPTION OF THE INVENTION

Because hyper-sulfated proteoglycans are found in association with all forms of amyloid, HSPG is useful as a target for imaging of amyloid for diagnostic or monitoring purposes and as a target for associating amyloid with a chemical compound that is effective in the treatment of amyloid.

In one embodiment, the invention is a method for targeting amyloid in the body of an individual suffering, or suspected of suffering, from a bodily disorder associated with amyloidosis. According to this embodiment of the invention, a ligand that specifically binds to a hyper-sulfated proteoglycan associated with amyloid is systemically introduced into the body of an individual. The ligand is permitted to bind to hyper-sulfated proteoglycans within the body of the individual.

The targeting of the hyper-sulfated proteoglycans may be used for diagnostic purposes. For example, the ligand may be, or may contain or be attached to, a label or marker that is detectable. In this way, upon creating an image of the body or of a body part, an image of the deposition pattern of the ligand, and thus of amyloid, in the body or body part may be obtained.

The targeting of the hyper-sulfated proteoglycans may be used for therapeutic purposes. For example, an antibody, or immunogenic antibody fragment, that specifically binds to a hyper-sulfated proteoglycan may be targeted to amyloid to form a complex. The opsonizing effect of such complexes is useful in treating amyloid-related diseases and conditions.

The method of the invention utilizes a ligand that binds to hyper-sulfated proteoglycans. As used herein, the term “hyper-sulfated” when referring to a proteglycan other than dermatan sulfate and chondroitin sulfate, means a sulfated proteoglycan that contain disaccharide units that contain more than two sulfate groups. With regard to dermatan sulfate and chondroitin sulfate, “hyper-sulfated” means containing two or more sulfate groups, such as at any two of positions 2, 4, and 6. As with other proteoglycans, the hyper-sulfated dermatan sulfate or chondroitin sulfate, of course, may contain disaccharide units that are tri-sulfated or may contain more than three sulfate groups.

The ligand that binds to the hyper-sulfated proteoglycans may be an antibody or may be an “immunoglobulin polypeptide”, that is a molecule that is derived from native immunoglobulins (e.g., antibodies) that has specific immunoreactive activity against a particular target. Antibodies are typically tetramers of immunoglobulin polypeptides. As used herein, the term “antibody” also refers to a protein that is substantially encoded by immunoglobulin genes.

Immunoglobulin genes include those coding for the light chains, which may be of the kappa or lambda types, and those coding for the heavy chains. The immunoglobulins may exist in a variety of fragment forms including, for example, Fv, Fab, F (ab'), F (ab')2, scFv (single chain Fv), and other fragments, as well as single chains. Examples of ligands include scFv selected for their reactivity with proteoglycans isolated from bovine kidney, human skeletal muscle, or human lung. Specific examples include the scFv designated NS4F5 that binds hypersulfated HS and the scFv GD3G7 that binds hypersulfated CS and DS.

Single-chain antibodies, in which genes for a heavy chain and a light chain are combined into a single coding sequence, may also be used. Immunoglobulin polypeptide also encompasses a truncated immunoglobulin chain, for example, a chain containing less constant region domains than in the native polypeptide. Such truncated polypeptides can be produced by standard methods such as introducing a stop codon into the gene sequence 5′ of the domain sequences to be deleted. The truncated polypeptides can then be assembled into truncated antibodies. Antibodies as used herein also include bispecific antibodies. If desired, a polypeptide fragment containing only a portion of a primary immunoglobulin structure may be produced. For example, it may be desirable to produce immunoglobulin polypeptide fragments that possess one or more immunoglobulin activities in addition to, or other than, antigen recognition, such as complement fixation.

Alternatively, the ligand may be a polypeptide or a small compound that binds to the hyper-sulfated proteoglycan. Methods for identifying such small compound ligands are disclosed in Kisilevsky, U.S. Pat. No. 5,164,295, which is incorporated in its entirety herein by reference.

In accordance with the method of the invention an amount of ligand sufficient to be distributed throughout the body and to bind to hyper-sulfated proteoglycans of amyloid present in the body is administered. Such administration may be by any means by which the ligand may be distributed throughout the body. Oral administration may be suitable but may result in destruction of the ligand due to digestive processes, especially in the case of polypeptide-based ligands. Consequently, parenteral administration, such as intravenous administration, is preferred. Other routes of parenteral administration include subcutaneous, intradermal, and intramuscular. If treatment or diagnosis of the central nervous system is desired, the delivery of antibodies, immunoglobulin polypeptides, non-immunoglobulin polypeptides, and small molecules may be delivered to the central nervous system utilizing techniques designed to overcome the blood-brain barrier, such as through liposomal or micellar delivery to the desired site or by administration directly into the cerebrospinal fluid.

The invention provides compositions for parenteral administration which contain a solution or suspension of the ligand in a pharmaceutically acceptable carrier, preferably an aqueous carrier. A variety of aqueous carriers may be used, e.g., water, buffered water, 0.4% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, or triethanolamine oleate.

The concentration of the ligand in the composition of the invention may vary widely, such as from less than about 0.5%, typically from 10-15%, to as much as 50% or more by weight, and will be selected primarily by such variables as fluid volumes and viscosities in accordance with the particular mode of administration selected.

The method of the invention may be used for therapeutic purposes, that is to bind amyloid, particularly hyper-sulfated proteoglycans of amyloid, to form a complex such that the complex, or the amyloid containing such complex, is recognized as “foreign” by the immune system, which in turn enhances phagocytosis of the amyloid. According to this method of the invention, an amount of the ligand is administered, systemically or locally, that opsonizes the amyloid, thus resulting in an impairment of further amyloid deposition and, preferably, also a reversal of the accumulation of amyloid in amyloid deposits.

The method of the invention may be used for diagnostic purposes, that is to provide a means for detection, and preferably quantification, of amyloid in the body or in particular portions of the body, such as in an organ like liver, lung, brain, heart, and pancreas. The diagnostic method of the invention may be used to diagnose that a person is suffering from an amyloid-related disorder, to determine the severity of an amyloid-related disorder, or to evaluate response to therapy in an individual suffering from an amyloid-related disorder.

The invention is further illustrated with the following non-limiting examples.

### Example 1

### Ligand for Hyper-Sulfated Proteoglycans

Two scFv reactive with hyper-sulfated disaccharides, designated NS4F5 and GD3G7, were selected from the Nissim scFv Phage display library according to established methods, as described in Smits et al, Methods in Enzymology, 416:61-87 (2006). The scFv were purified as follows: Periplasmic extracts from bacteria producing scFv were received frozen. The extracts were thawed and dialyzed versus 10 mM sodium phosphate pH 7.6 and 0.15 M NaCl (PBS). The samples were adjusted to 0.3 M NaCl and incubated batchwise with 0.2 mL of 1:1 suspension of Ni-NTA Agarose beads (Quiagen) overnight at room temperature with continuous end over end mixing. Beads were washed 3 times with wash solution (0.3 M NaCl, 50 mM sodium phosphate pH 7.5, 20 mM imidazole). The beads were transferred to a 1 mL syringe barrel plugged with glass wool and the scFv eluted with ˜400 μL elution buffer (0.3 M NaCl, 50 mM sodium phosphate pH 7.5, 250 mM imidazole). Samples were dialyzed versus PBS, clarified by centrifugation and stored at 4° C. Purity was assessed by SDS-PAGE stained with Coomassie blue.

### Example 2

The in vivo reactivity of the scFv of Example 1 with amyloid was studied using a murine model of severe systemic Amyloid A (“AA”) amyloidosis. The transgenic experimental murine model of AA amyloidosis uses mice that express the human interleukin-6 (IL-6) transgenic under the constitutive control of the major histocompatibility class 2H-2^{d} promoter (H-2/huIL-6 mice). These animals spontaneously develop amyloid by age 5 month and die of the disease at about 8 to 9 months of age. However the process of amyloid deposition can be significantly accelerated by intravenously injecting 8 week-old H-2/huIL-6 mice with an extract of amyloid enhancing factor (AEF), a solution of preformed fibrils isolated for the tissues AA amyloid deposits in the liver, spleen, pancreas, and kidney within 8 wk post injection.

### Example 3

### Radiolabeling of scFv

The scFv of Example 1 (40 μg) were labeled with 2 mCi of reductant-free ¹²⁵I (Perkin Elmer) using limiting amounts of Chloramine T. The labeled reagents were suspended in PBS containing 5 mg/ml of bovine serum albumin (BSA/PBS) and unbound isotope and protein aggregates removed by size-exclusion liquid chromatography through an Ultrogel AcA34 column (Amersham Pharmacia). Fractions containing scFv monomer were pooled for imaging experiments. The radiochemical yield was ˜50%, providing a specific activity of ˜25 μCi/μg. ¹²⁵I-labeled scFv was subjected to SDS/PAGE (10% gels) in the presence and absence of a reducing agent and analyzed with a Cyclone phosphor-imager.

### Example 4

### MicroSPECT/CT Imaging of the Radioiodinated scFv in Mice

To study the in vivo localization of radiolabeled scFv of Example 3, each scFV was injected iv in the lateral tail vein of an AEF-treated H-2/huIL-6 and a healthy (amyloid free) Balb/c mouse. Each mouse received ¹²⁵I-labeled scFv (˜10 μg, 150 μCi per mouse) iv in the lateral tail vein. After 1 hr the mice were sacrificed by isoflurane overdose and SPECT/CT images were acquired. To provide vascular contrast-enhancement in the CT images, all mice were given a 200 μL iv doses of Fenestra VC™ (Advanced Research Technologies, Montreal, Canada) 15 min prior to scanning.

SPECT data were collected with a microCAT II+SPECT dual modality imaging platform (Siemens Preclinical Solutions, Knoxville, Tenn.) capable of sub-millimeter spatial resolution when equipped with a 0.5 mm-pore diameter pinhole collimator. When imaging, the 2 detectors (composed of a 50 mm-diameter Hamamatsu R2486-02 multi-anode photo-multiplier tube coupled to a 1×1×8 mm CsI (Tl) crystal array arranged on a 1.2 mm² grid) were positioned ˜45 mm from the center of rotation. Each SPECT dataset comprised 45 projections collected over 360° during the course of ˜50 min. Images were reconstructed using an implementation of the expectation maximization-maximum likelihood (EM-ML) algorithm.

After collection of SPECT data, high-resolution CT images were obtained using the same machine. The microCAT II scanner has a circular orbit cone beam geometry, equipped with a 20-80 kVp microfocus x-ray source, and captures a 90 mm×60 mm field of view using a 2048×3072 CCD array detector, optically coupled to a minR phosphor screen via a fiber-optic bundle. Each CT dataset, composed of 360 projections at 1° azimuths, was acquired in 8 min. Images were reconstructed in real-time on isotropic 77-μm voxels using an implementation of the Feldkamp backprojection algorithm.

To facilitate co-registration of the reconstructed SPECT and CT images, Co-57 sealed sources were placed on the imaging bed. The microSPECT and CT datasets were visualized and co-registered manually with a 3-D image analysis software package (Amira, Version 3.1: Mercury Computer Systems).

### Example 5

### Biodistribution

Samples of various tissues including skin, muscle, liver, spleen, pancreas, kidney, upper and lower stomach, upper, mid and lower intestine, heart, lung, and tongue were harvested from AA amyloid-bearing and control mice injected with ¹²⁵I-scFv. The tissues were placed into tared vials, weighed, and the radioactivity associated with each sample measured. The primary index values were expressed as % injected dose/g tissue (% ID/g).

### Example 6

### Autoradiography 6 micron-thick sections cut from formalin-fixed, paraffin-embedded blocks of tissue obtained from mice sacrificed 4 h post-injection of ¹²⁵I-scFv were placed on Probond microscope slides (Fisher Scientific), dipped in NTB-2 emulsion (Eastman Kodak), stored in the dark, and developed after a 4-d exposure. The sections were counter-stained with hematoxylin and eosin (H&E), cover-slipped using Permount (Fisher Scientific), and examined by light microscopy. In addition, consecutive slides were stained with alkaline Congo red and viewed under cross-polarized illumination. Digital microscopic images were acquired using a cooled-SPOT camera and evaluated using an image analysis software package (Image Pro Plus, Media, Cybernetics).

### Example 7

### Results

In disease-free mice without amyloid, ¹²⁵I-scFv tested bound to heparan sulfate naturally occurring in the renal tubules as well as glomerular tufts and capsules. In addition, catabolism and dehalogenation of the scFv in the kidney liberated free ¹²⁵[I] iodide that accumulated in the stomach at 4 h post-injection as evidenced in microSPECT images.

In contrast, when ¹²⁵I-labeled scFv was injected into mice with AA amyloid, the radioisotope deposited in the liver, kidney, heart, pancreas, spleen, and intestine—sites of amyloid deposition in this animal model. Microautoradiogaphy revealed that in the presence of amyloid, the hypersulfated disaccharide-reactive scFv preferentially associated with the pathologic amyloid deposits in all organs. These data indicate that even though heparan sulfate is expressed in normal tissue, scFv reactive with hyper-sulfated forms of amyloid, such as heparan sulfate, chondroitin sulfate, and dermatin sulfate, bind specifically to amyloid deposits and are suitable for imaging and treating amyloid diseases.

Further modifications, uses, and applications of the invention described herein will be apparent to those skilled in the art. It is intended that such modifications be encompassed in the following claims.

1. A method for targeting amyloid in an individual suspected of suffering from amyloidosis comprising administering to the individual a composition containing a ligand that specifically binds to a hyper-sulfated proteoglycan and permitting the ligand to bind to hyper-sulfated proteoglycans within the body of the individual, thereby indirectly binding to amyloid.

2. The method of claim 1 wherein the ligand is an antibody or antibody fragment.

3. The method of claim 1 which further comprises imaging the body of the individual and thereby determining if the ligand has localized to one or more sites within the body.

4. The method of claim 1 wherein the ligand is administered intravenously.

5. The method of claim 1 wherein the ligand is administered in a pharmaceutical composition comprising a carrier and the ligand.

6. The method of claim 5 wherein the concentration of the ligand in the pharmaceutical composition is between 0.5% to 50% by weight.
